(19) United States
(12) Patent Application Publication
(10) Pub. No.: US 2011/0125151 A1
(43) Pub. Date: May 26, 2011

(54) HIGH FREQUENCY SURGICAL DEVICE

(76) Inventors: Timo Strauss, Berlin (DE); Stefan Schiddel, Potsdam (DE); Uwe Fischer, Berlin (DE)

(21) Appl. No.: 12/624,492

(22) Filed: Nov. 24, 2009

(51) Int. Cl. A61B 18/18

(52) U.S. Cl. 606/37

(57) ABSTRACT

The invention relates to a high frequency surgical device for generating high frequency energy for cutting and/or coagulating biological tissues, comprising at least one resonance circuit with at least two switches, through which an electrical connection between the resonance circuit and an electrical energy source can be switched respectively during operation, in order to supply electrical energy to the resonance circuit during operation at least for periods of time, and with at least one control device associated with the switches through which control device the switches can be switched independently from another.

The invention relates to a high frequency surgical device for generating high frequency energy for cutting and/or coagulating biological tissues.

High frequency or HF surgical devices of this type have been known in the art for quite a while and are also designated as HF generators. The HF surgical device generates HF output energy for cutting and/or coagulating biological tissues. Various mono-polar or bi-polar instruments can be connected to the HF surgical device, which instruments introduce the HF output energy into the biological tissue of a patient to be treated. In or at the tissue, the HF energy causes the desired electrosurgical cutting or coagulating.

In order to generate the high frequency output energy required for HF surgery, namely high frequency AC power in the HF surgical device, typically a parallel resonance circuit is provided. The resonance circuit is charged with electrical energy through a DC power source and generates an electrical oscillation, which can be tapped as high frequency AC power. Through selecting the capacitor and the coil for the resonance circuit, the frequency of the output energy is determined. In order to sustain the operating oscillations, the exact amount of energy is provided to the parallel resonance circuit proximal to the maximum of the positive or negative half cycle of the HF voltage, as it was previously extracted from the resonance circuit through the HF surgical application and the natural attenuation of the resonance circuit.

The resonance circuit is connected to an energy source through a switch, e.g. a transistor, for a short period of time in order to supply energy to it. The correct point in time for switching the transistors in order to sustain the oscillation in the resonance circuit can be determined e.g. by a zero transition detector. Thus, the HF surgical device is suitable for a broad load range and rather tolerant with respect to changes of the operating frequency.

It is a problem of the recited circuit that the thermal load on the switch, e.g. the transistor, can be very high for periods of time. The thermal load can be caused by a high current, e.g. when charging the capacitor of the resonance circuit for the first time. Furthermore, the transistor is not always completely set to maximum due to its relatively short switching period power-on time, so that the power dissipation at the transistor can be rather high. In order to reduce the current, it is possible to use two parallel transistors. However, the total current is not evenly divided between the transistors due to technology based differences, like e.g. slightly different gate drain capacity. The uneven current distribution in turn leads to a problematic uneven thermal loading of the transistors and also to a degradation of the signal pattern of the oscillation.

Therefore, it is the object of the invention to provide an improved high frequency surgical device, which overcomes the recited problems.

The object is accomplished through the high frequency surgical device according to claim 1. The high frequency surgical device comprises at least one resonance circuit and at least two switches, through which an electrical connection between the resonance circuit and an electrical energy source can be switched, in order to provide the resonance circuit with electrical energy during operation, at least for particular time periods. Furthermore, the high frequency surgical device according to the invention comprises at least one control device associated with the switches, through which the switches can be switched independently from one another.

The solution according to the invention has the advantage that the thermal load can be distributed over two or more switches. Since the switches can be controlled by the control unit independently from one another, they can be switched as required. Thus, an individual control of the resonance circuit can be provided as a function of the load.

The solution according to the invention can be supplemented by additional advantageous embodiments. Some of these embodiments are described infra.

Thus, the control device can be configured, so that it alternatively switches the switches in a first operating mode. This has the advantage that the thermal load on each switch is reduced, since the control frequency of each switch is reduced. In a configuration with two switches, they are controlled in an alternating manner. In a configuration with n switches only the first switch is switched accordingly at the maximum of the first half wave in the resonance circuit, at the maximum of the next half wave only the second switch is switched, etc. At the maximum of the n+1^{st} half wave, in turn only the first switch is switched. The power dissipation and thus the heat load are evenly distributed over two or more transistors. The first operating mode of the control device can certainly also be the only operating mode, thus the switches can be permanently controlled in alternating manner. Thus, the control devices can comprise e.g. a control circuit with switchover, or also separate control circuits for each switch.

In another advantageous embodiment, the control device can be configured, so that it substantially switches the switches in parallel in another operating mode. This has the advantage that a switchover between different operating modes can be provided in order to be able to optimally adjust the control scheme of the switches to different operating phases of the high frequency surgical device. Thus, the switches can e.g. be switched in parallel in a first oscillation onset phase in order to distribute the high current flow over several switches. When the resonance circuit has reached resonance and the current flow is reduced, the switches can be switched alternatively in order to facilitate a more homogeneous oscillation in the resonance circuit. Furthermore, it is also possible to switch the switches in parallel, when the HF output signals of the high frequency surgical device are modulated when the thermal load of the switches is particularly high. When the HF output signals are not modulated, the switches can be switched e.g. alternatively.

Furthermore, the control device can be configured, so that it only switches one switch in an additional operating mode. Thus, it is possible e.g. for non-modulated output signals of the HF surgical device to select this operating mode and to only activate one switch. Thus, the energy supply for the resonance circuit is also switched through the same switch, so that minor technology related differences between the switches, like e.g. for transistors, are insignificant. For modulated output signals, where the thermal load for a particular switch can be excessively high, a switchover to another operating mode can be switched, in which the load is distributed over several switches.

In an advantageous embodiment of the invention, the resonance circuit can be configured as a parallel resonance circuit.

In order to be able to control the high frequency surgical device in an optimum manner, it can comprise a measurement device, which detects at least one operating parameter, like e.g. time, temperature, and presence of a modulation of the HF output signal, current, voltage or power. Thus, the control device can be configured so that it activates different operating modes during operation as a function of at least one of the operating parameters. Thus, the control device can e.g. alternate from an operating mode starting at a predetermined temperature, in which operating mode the switches are controlled alternatively, to an operating mode where the switches are controlled in parallel.

The invention with its advantageous embodiments facilitates an optimum control of the resonance circuit of the HF surgical device with an adaptation e.g. to a HF output signal, efficiency, thermal loss, signal form and/or harmonic wave suppression. These parameters can be determined by the control device or at other locations in the HF surgical device and can be used by the control device for determining the switching times for the switches.

For a switching without significant time losses, the switches can be configured as transistors, in particular field effects or bipolar transistors.

In order to determine the optimum point in time for switching, the high frequency surgical device can have a signal conductor, which connects the control device to the resonance circuit for signal transfer. The control device can monitor the oscillations through the signal conductor. The control device can be configured, so that it operatively captures at least one parameter of the electrical oscillation in the resonance circuit, and so that it switches the switches as a function of the parameter. Thus, the control device can determine e.g. the point in time of maxima of the positive or negative half waves and can switch the switches accordingly.

In another advantageous embodiment, the high frequency surgical device can comprise an intermediate circuit and a patient circuit, galvanically separated from the intermediate circuit through at least one transformer, and the resonance circuit can comprise the winding of the transformer disposed within the intermediary circuit. Thus, the resonance circuit which is connected to the energy for certain periods of time is galvanically separated from the patient circuit, and the transformer simultaneously forms the inductivity of the resonance circuit, so that no additional component is required.

The invention is subsequently described with reference to the embodiments illustrated in the drawing figure. The various features can be combined with one another at will as it is also the case for the embodiments described supra.

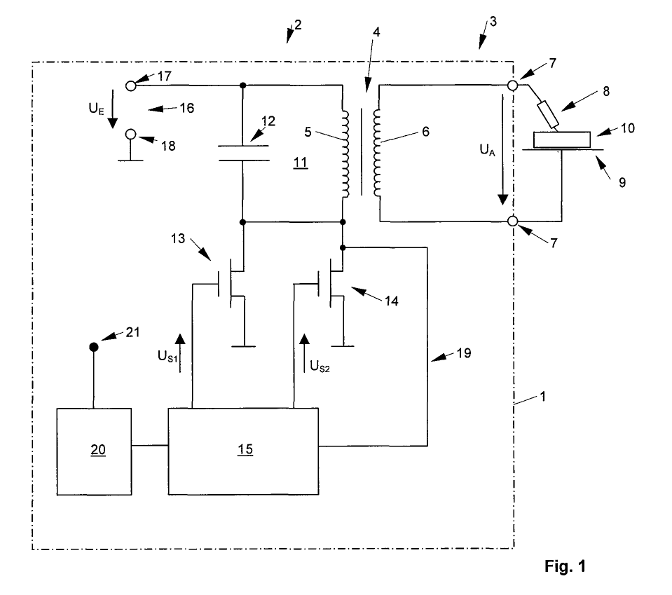

FIG. 1 illustrates a schematic of an exemplary embodiment of a high frequency surgical device according to the invention;

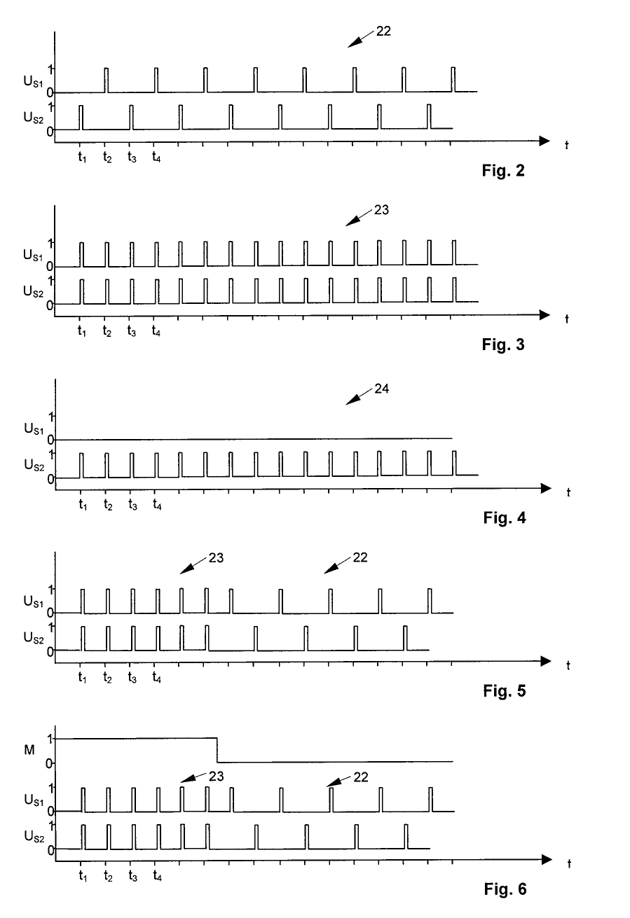

FIG. 2 illustrates a schematic of a first circuit diagram for the high frequency surgical device in FIG. 1; and

FIGS. 3-8 illustrate additional circuit diagrams for the high frequency surgical device in FIG. 1.

Initially, the configuration of a high frequency surgical device according to the invention is described with reference to the schematic illustration in FIG. 1.

In the embodiment of FIG. 1, the high frequency surgical device, which is illustrated in a highly simplified manner, comprises an intermediary circuit 2 and a patient circuit 3, which are galvanically separated from another through a transformer 4. Certainly, the high frequency surgical device 1 also comprises a grid circuit, which is galvanically separated from the intermediary circuit 2, through which grid circuit line voltage is conducted into the HF surgical device 1. For reasons of clarity, the line circuit is not illustrated in FIG. 1.

The transformer 4 comprises a first winding 5 associated with the intermediary circuit 2 and a second winding 6 associated with the patient circuit 3.

Two output contacts 7 are disposed in the patient circuit 3, at which an output voltage U_{A} of the high frequency surgical device 1 is applied during operation, and can be contacted. At the output contacts 7, a surgical instrument 8 is connected on one side in FIG. 1 and a neutral electrode 9 is connected on the other side, through which biological tissue 10 of a patient can be coagulated in a known manner, and/or can be cut electrosurgically.

The intermediate circuit 2 includes a DC power source 16, a resonance circuit 11, comprised of the second winding 5 of the transformer 4 and a capacitor 12, two transistors 13, 14 and a control unit 15.

The DC power source 16 provides an input voltage U_{E} between its two poles 17, 18. The one pole 17 of the AC power source 16 is electrically connected to the one side of the resonance circuit 11, the other pole 18 is connected to the other side of the resonance circuit 11 through the transistors 13, 14 connected in parallel as will be described in more detail infra.

The resonance circuit in FIG. 1 is a parallel resonance circuit, since its capacitor 12 and its inductivity in the form of a winding 5 are disposed in parallel to one another. The resonance circuit 11 is connected to the pole 17 on one side and to the two transistors 13, 14 connected in parallel on the other side. In the embodiment in FIG. 1, the two transistors 13, 14 are configured as field effect transistors. Alternatively e.g., also transistors of another type like e.g. bipolar transistors can be used. The transistors include a source-, a drain- and a gate contact. The source contacts are electrically connected respectively with the resonance circuit 11. The drain contacts of the transistors 13, 14 are respectively connected to the second pole 18 of the DC power source 16. The gate contacts of the transistors 13, 14 are respectively electrically connected to the control unit 15 independently from one another. The control unit 15 is additionally signal coupled to the resonance circuit 11 through a separate signal conductor 19.

Eventually, the surgical device 1 also includes a measuring unit 20 electrically connected with the control unit 15, which measuring unit comprises a temperature sensor 21.

Subsequently, the function of the HF surgical device 1 according to the invention will be described.

During operation of the HF surgical device 1, an electrical oscillation is generated in the parallel resonance circuit 11, which oscillation is provided in the form of an AC voltage. The AC voltage is transmitted from the intermediary circuit 2 through a transformer 4 to the patient circuit 3. In the patient circuit 3, the AC voltage is provided as an output voltage U_{A} to the output contacts 7 and can be contacted for electrosurgical applications as described supra.

In order to generate the high frequency output voltage in the parallel resonance circuit 11, the resonance circuit 11 is connected to the DC voltage source 16 for oscillation buildup. In order to sustain the oscillations after buildup, energy from the DC voltage source 16 is provided to the resonance circuit 11 proximal to the maximum, thus the reversal point of the positive or negative half wave of the oscillation. The exact amount of energy is provided, which has been dissipated by the load, this means the surgical application at the biological tissue 10 and through the power losses.

The temporary energy supply to the resonance circuit 11 is implemented through the transistors 13, 14. Each of the two transistors 13, 14 disposed in parallel to one another is a switch which connects or disconnects the connection of the resonance circuit 11 to the second pole 18 of the DC current source 16. The advantage of using transistors as switching devices is that they can switch very quickly. The transistors 13, 14 are switched independently from one another through the control unit 15. The control unit 15 activates the transistors 13, 14 respectively through a switching voltage U_{S1}, U_{S2}, which connects the switching unit 15 to the base contact of the transistor 13, 14. Within the control unit 15, the switching voltage U_{S1}, U_{S2} is amplified through an amplifier unit (not shown), so that the switching voltages U_{S1}, U_{S2} are large enough to switch the transistors 13, 14. When a sufficient amount of energy has been provided to the resonance circuit 11, the control unit 15 deactivates the switching voltages U_{S1}, U_{S2}, and one or both transistors separate the connections of the resonance circuit 11 to the pole 18 of the DC voltage source 16.

In order to be able to determine the correct point in time for switching, the switching unit 15 is connected for signal transmission to the resonance circuit 11 through the signal conductor 19. Thus, the switching unit 15 can determine the zero point of the oscillation in the resonance circuit 11 through an integrated zero point detector, and can thus determine the optimum point in time for switching the transistors 13, 14.

It is a substantial advantage of the present invention that the transistors 13, 14 can be switched independently from one another and supply energy to the resonance circuit 11 independently from one another. During operation of the high frequency surgical device according to the invention, the transistors 13, 14 can be switched at will.

Various switching patterns are subsequently described with reference to FIGS. 2-8. The switching patterns illustrate the time based activation of the transistors 13, 14 through the switching unit 15 as a function of the switching voltages U_{S1}, U_{S2}.

The diagrams in FIGS. 2-8 show the value of the switching voltages U_{S1}, U_{S2} in a simplified manner as a value of 1 when the switching voltage is activated by the control unit 15, or they show it with the value 0 when the switching voltage is deactivated. The illustrated points in time t₁, t₂, t₃, etc. are the points in time at which energy has to be provided to the resonance circuit, in order to generate the desired oscillation. The points in time t₁, t₂, t₃, etc. are determined by the control unit 15 as described supra. Their frequency is substantially predetermined through the configuration of the resonance circuit based on the desired frequency of the output voltage U_{A}. The activation duration illustrated in the diagrams of FIGS. 2-8 is only used for illustration purposes and is not realistic.

FIG. 2 shows the operation of the HF surgical device according to the invention in a first operating mode 22, in which the two transistors 13, 14 are switched alternatively, this means in periodic alternation. Thus, each of the two transistors 13, 14 is only activated at each second point in time t₁, t₂, t₃, so that the transistors 13, 14 respectively can cool down for a longer period of time.

FIG. 3 illustrates the operation of the HF surgical device 1 according to the invention in another operating mode 23, in which the two transistors 13, 14 are switched in parallel. Thus, a high current that causes a high thermal load such as during oscillation buildup of the resonance circuit 11 can be distributed over both transistors 13, 14.

FIG. 4 illustrates the operation of the HF surgical device 1 according to the invention in another operating mode 24, in which only the transistor 13 is activated by the switching voltage U_{S1}. The other transistor 14 is not activated in this operating mode. Certainly, only the transistor 14 can be switched through the switching voltage U_{S2} in a similar operating mode. This operating mode has the advantage that the same transistor 13, 14 is used all the time. Thus, slight technological differences of the transistor, like e.g. a slightly different gate drain capacity, do not become effective. These differences can impact the course of the oscillation negatively.

The various operating modes 22, 23, 24 can be combined with one another to optimally control the HF surgical device 1. As a function of various operating parameters of the HF surgical device 1, the one or the other operating mode can be advantageous. The operating parameters are e.g. time, temperature, presence of a modulation of the HF output signal, output current, output voltage or output power.

In order to detect the operating temperature, the HF surgical device 1 in FIG. 1 comprises the measurement unit 20, which is signal connected to the control unit 15. The measurement unit 20 in FIG. 1 is connected to the temperature sensor 21 for detecting the temperature in the portion of the transistors 13, 14. Additional operating parameters can be determined in a known manner. The measurement unit 20 transmits the operating parameters or the signals representing the operating parameters to the control unit 15. The control unit 15 can alternate between different operating modes when exceeding or falling below certain threshold values for the operating parameters. Such switching between different operating modes is subsequently described in an exemplary manner with reference to FIGS. 5-8.

FIG. 5 shows a switching from the operating mode 23 with parallel control of the transistors 13, 14 to the operating mode 22 with alternating control. This is advantageous in particular when starting the HF surgical device 1, thus during oscillation buildup of the resonance circuit 11, since the high initial thermal load is divided by a high current between both transistors 13, 14. Subsequently, when the oscillation in the resonance circuit 11 has built up and the current through the transistors 13, 14 is reduced, a switching occurs to the alternating operating mode 22. The switching point in time can be controlled e.g. time based.

FIG. 6 like FIG. 5 also shows the switching between the operating modes 22, 23. In addition to the switching voltages U_{S1}, U_{S2}, also a signal M is illustrated in the diagram in FIG. 6, which indicates if the output signal is modulated or not. The illustrated modulation signal SM has a value of 1 when a modulated output signal is present and has a value 0 when no modulation is present. The parallel operating mode 23 is used when a modulated output signal is present; the alternating operating mode 22 is used for a non-modulated output signal. This switching is advantageous, since a high thermal load for the transistors 13, 14 is provided for modulated output signals.

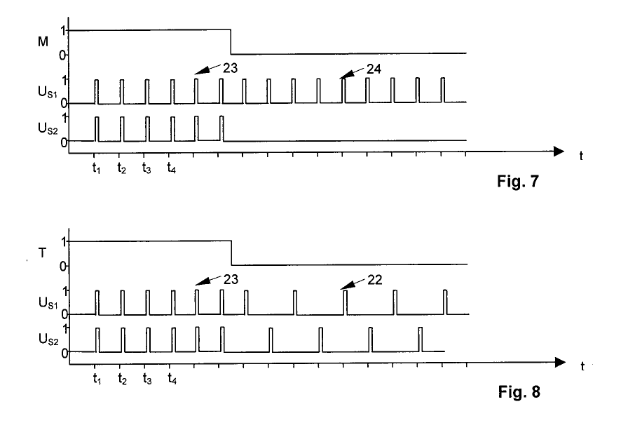

For the control system illustrated in FIG. 7, the switching also occurs as a function of the modulation of the output signal, but here, a switching occurs from the parallel operating mode to the simple operating mode 24.

Eventually, FIG. 8 in turn illustrates the switching from the parallel operating mode 23 to the alternating operating mode 22. However, the switching occurs here as a function of the temperature T. The illustrated temperature signal T has a value of 1 above a predetermined temperature threshold value and a value of 0 below the temperature threshold value.

Certainly, also switching between different operating modes is possible as a function of other operating parameters.

1. A high frequency surgical device for generating high frequency energy for cutting and/or coagulating biological tissues, comprising at least one resonance circuit with at least two switches, through which an electrical connection between the resonance circuit and an electrical energy source can be switched respectively during operation, in order to supply electrical energy to the resonance circuit during operation at least for periods of time, and with at least one control device associated with the switches through which control device the switches can be switched independently from another.

2. A high frequency surgical device according to claim 1, wherein the control device is configured so that it substantially switches the switches alternatively in a first operating mode.

3. A high frequency surgical device according to claim 1, wherein the control device is configured, so that it switches the switches substantially in parallel in another operating mode.

4. A high frequency surgical device according to claim 2, wherein the control device is configured, so that it only switches one switch in another operating mode.

5. A high frequency surgical device according to claim 1, wherein the resonance circuit is configured as a parallel resonance circuit.

6. A high frequency surgical device according to claim 1, wherein the high frequency surgical device comprises a measurement device, which detects at least one operating parameter like time, temperature, presence of a modulation of the HF output signal, current, voltage or power, and the control device is configured, so that it activates different operating modes during operation as a function of at least one operating parameter.

7. A high frequency surgical device according to claim 1, wherein the switches are configured as transistors, in particular field effect or bipolar transistors.

8. A high frequency surgical device according to claim 1, wherein the high frequency surgical device comprises a signal conductor, which connects the signal of the control device to the resonance circuit.

9. A high frequency surgical device according to claim 1, wherein the control device is configured, so that it detects at least one parameter of the electrical oscillation in the resonance circuit and switches the switches as a function of the parameter.

10. A high frequency surgical device according to claim 1, wherein the high frequency surgical device comprises an intermediary circuit and a patient circuit separated from the intermediary circuit through at least one transformer, and wherein the resonance circuit comprises the winding of the transformer disposed within the intermediary circuit.

11. A high frequency surgical device according to claim 3, wherein the control device is configured, so that it only switches one switch in another operating mode.

12. A high frequency surgical device according to claim 1, wherein the resonance circuit is configured as a parallel resonance circuit.

13. A high frequency surgical device according to claim 1, wherein the high frequency surgical device comprises a measurement device, which detects at least one operating parameter like time, temperature, presence of a modulation of the HF output signal, current, voltage or power, and the control device is configured, so that it activates different operating modes during operation as a function of at least one operating parameter.

14. A high frequency surgical device according to claim 1, wherein the switches are configured as transistors, in particular field effect or bipolar transistors.

15. A high frequency surgical device according to claim 1, wherein the high frequency surgical device comprises a signal conductor, which connects the signal of the control device to the resonance circuit.

16. A high frequency surgical device according to claim 1, wherein the control device is configured, so that it detects at least one parameter of the electrical oscillation in the resonance circuit and switches the switches as a function of the parameter.

17. A high frequency surgical device according to claim 1, wherein the high frequency surgical device comprises an intermediary circuit and a patient circuit separated from the intermediary circuit through at least one transformer, and wherein the resonance circuit comprises the winding of the transformer disposed within the intermediary circuit.

18. A high frequency surgical device according to claim 1, wherein the resonance circuit is configured as a parallel resonance circuit.

19. A high frequency surgical device according to claim 1, wherein the resonance circuit is configured as a parallel resonance circuit.

20. A high frequency surgical device according to claim 1, wherein the high frequency surgical device comprises a measurement device, which detects at least one operating parameter like time, temperature, presence of a modulation of the HF output signal, current, voltage or power, and the control device is configured, so that it activates different operating modes during operation as a function of at least one operating parameter.
